(19) United States
(12) Patent Application Publication
(10) Pub. No.: US 2023/0196511 A1
(43) Pub. Date: Jun. 22, 2023

(54) IMAGE PROCESSING APPARATUS, IMAGE CAPTURING APPARATUS, IMAGE CAPTURING SYSTEM, AND METHOD

(71) Applicant: CANON KABUSHIKI KAISHA, Tokyo (JP)

(72) Inventor: Chiaki MIKAWA, Kanagawa (JP)

(21) Appl. No.: 18/063,109

(22) Filed: Dec. 8, 2022

(30) Foreign Application Priority Data
Dec. 20, 2021 (JP) 2021-206263

(51) Int. Cl. G06T 3/60; G06T 7/00; G06F 3/14; G16H 30/40

(52) U.S. Cl. CPC G06T 3/60; G06T 7/0012; G06F 3/14; G16H 30/40; G06T 2207/30036

(57) ABSTRACT

An image processing apparatus comprising one or more processors and/or circuitry which functions as: an acquisition unit that acquires an image file including first image data of an intraoral photograph, site information indicating a site in an oral cavity, and mirror use information indicating whether or not a mirror was used at a time of photography; and a processor that determines whether or not it is necessary to perform at least one of horizontal reversal processing and vertical rotation processing on the first image data in the acquired image file based on the site information and the mirror use information, and performs the processing determined to be necessary.

### BACKGROUND OF THE INVENTION

### Field of the Invention

The present invention relates to an image processing apparatus, image capturing apparatus, image capturing system, and method, and more particularly to technology for processing captured intraoral images.

### Description of the Related Art

In oral surgery and dentistry, medical personnel take intraoral photographs for intraoral diagnostic and documentation purposes. By using the intraoral photographs, it becomes possible to formulate treatment policies and plans, and to evaluate post-treatment. There are 5-, 9-, 12-sheet methods, and the like, that take photographs of the patient's oral cavity from multiple directions, and especially the five-sheet method is widely used as a method capable of recording the state of the oral cavity with a small number of photographs. Intraoral photographs taken with visible light have the advantage that they can be used for diagnosing and recording the state of gums, the color of teeth, and the like.

On the other hand, X-ray photography is sometimes used in another method for intraoral diagnostic and recording. Japanese Patent Laid-Open No. 2016-67532 discloses an image processing apparatus that has an image size adjustment unit and an image brightness adjustment unit in order to align the size and brightness of a plurality of X-ray images taken from multiple directions when combining and synthesizing them, and that adjusts the brightness of the X-ray images so as to match the mean of a histogram.

However, with the technology disclosed in Japanese Patent Laid-Open No. 2016-67532, although it is possible to align the brightness and size of the X-ray images, it is not possible to match how the patient's oral cavity actually looks when it is observed and how intraoral photographs look since the taking order of the intraoral photographs may be different between patients and some of the intraoral photographs may be taken using a mirror.

### SUMMARY OF THE INVENTION

The present invention has been made in consideration of the above situation, and make how a plurality of intraoral images look and how a patient's oral cavity actually looks when it is observed be the same.

According to the present invention, provided is an image processing apparatus comprising one or more processors and/or circuitry which functions as: an acquisition unit that acquires an image file including first image data of an intraoral photograph, site information indicating a site in an oral cavity, and mirror use information indicating whether or not a mirror was used at a time of photography; and a processor that determines whether or not it is necessary to perform at least one of horizontal reversal processing and vertical rotation processing on the first image data in the acquired image file based on the site information and the mirror use information, and performs the processing determined to be necessary.

Further, according to the present invention, provided is an image capturing apparatus comprising: an image sensor that takes intraoral photographs; and one or more processors and/or circuitry which functions as: a setting unit that sets site information indicating a site in an oral cavity, and mirror use information indicating whether or not a mirror was used at a time of photography; and transmission unit that transmits to an external device an image file in which the site information and the mirror use information set at the time of photography are appended to image data of each of the intraoral photographs taken by the image sensor.

Furthermore, according to the present invention, provided is an image capturing system comprising an image capturing apparatus and an image processing apparatus, wherein the image capturing apparatus comprises: an image sensor that takes intraoral photographs; and one or more processors and/or circuitry which functions as: a setting unit that sets site information indicating a site in an oral cavity, and mirror use information indicating whether or not a mirror was used at a time of photography; and transmission unit that transmits to the image processing apparatus an image file in which the site information and the mirror use information set at the time of photography are appended to first image data of each of the intraoral photographs taken by the image sensor, and the image processing apparatus comprises: one or more processors and/or circuitry which functions as: a receiving unit that receives the image file from the image capturing apparatus; and a processor that determines whether or not it is necessary to perform at least one of horizontal reversal processing and vertical rotation processing on the first image data in the received image file based on the site information and the mirror use information, and performs the processing determined to be necessary.

Further, according to the present invention, provided is an image processing apparatus comprising one or more processors and/or circuitry which functions as: an acquisition unit that acquires image data of a plurality of intraoral photographs; a determination unit that determines whether or not a site information indicating a site in an oral cavity, and mirror use information indicating whether or not a mirror was used at a time of photography are appended to the image data of each of the intraoral photographs acquired by the acquisition unit; an inference unit that, in a case where neither of the site information and the mirror use information is appended, infers the site and whether or not a mirror was used based on a characteristic of each of the intraoral photographs; and a memory that stores the site information and the mirror use information by appending them to the image data of each of the intraoral photographs based on the site and whether or not a mirror is used inferred by the inference unit.

Further, according to the present invention, provided is an image processing method comprising: acquiring an image file including image data of an intraoral photograph, site information indicating a site in an oral cavity, and mirror use information indicating whether or not a mirror was used at a time of photography; and determining whether or not it is necessary to perform at least one of horizontal reversal processing and vertical rotation processing on the image data in the image file based on the site information and the mirror use information, and performing the processing determined to be necessary.

Further, according to the present invention, provided is an image capturing method comprising: taking intraoral photographs; and setting site information indicating a site in an oral cavity, and mirror use information indicating whether or not a mirror was used at a time of photography; and transmitting to an external device an image file in which the site information and the mirror use information set at the time of photography are appended to image data of each of the intraoral photographs.

Further, according to the present invention, provided is an image capturing method comprising: in an image capturing apparatus, taking intraoral photographs; setting site information indicating a site in an oral cavity, and mirror use information indicating whether or not a mirror was used at a time of photography; and generating an image file in which the site information and the mirror use information set at the time of photography are appended to image data of each of the intraoral photographs, and in an image processing apparatus, determining whether or not it is necessary to perform at least one of horizontal reversal processing and vertical rotation processing on the image data in the image file based on the site information and the mirror use information, and performing the processing determined to be necessary.

Further, according to the present invention, provided is an image processing method comprising: acquiring image data of a plurality of intraoral photographs; determining whether or not a site information indicating a site in an oral cavity, and mirror use information indicating whether or not a mirror was used at a time of photography are appended to the image data of each of the intraoral photographs; inferring, in a case where neither of the site information and the mirror use information is appended, the site and whether or not a mirror was used based on a characteristic of each of the intraoral photographs; and storing the site information and the mirror use information by appending them to the image data of each of the intraoral photographs in a memory based on the inferred site and whether or not a mirror is used.

Further, according to the present invention, provided is a non-transitory computer-readable storage medium, the storage medium storing a program that is executable by the computer, wherein the program includes program code for causing the computer to execute the image processing method comprising: acquiring an image file including image data of an intraoral photograph, site information indicating a site in an oral cavity, and mirror use information indicating whether or not a mirror was used at a time of photography; and determining whether or not it is necessary to perform at least one of horizontal reversal processing and vertical rotation processing on the image data in the image file based on the site information and the mirror use information, and performing the processing determined to be necessary.

Further, according to the present invention, provided is a non-transitory computer-readable storage medium, the storage medium storing a program that is executable by the computer, wherein the program includes program code for causing the computer to execute the image capturing method comprising: taking intraoral photographs; and setting site information indicating a site in an oral cavity, and mirror use information indicating whether or not a mirror was used at a time of photography; and transmitting to an external device an image file in which the site information and the mirror use information set at the time of photography are appended to image data of each of the intraoral photographs.

Further, according to the present invention, provided is a non-transitory computer-readable storage medium, the storage medium storing a program that is executable by the computer, wherein the program includes program code for causing the computer to execute the image processing method comprising: in the image capturing apparatus, taking intraoral photographs; setting site information indicating a site in an oral cavity, and mirror use information indicating whether or not a mirror was used at a time of photography; and generating an image file in which the site information and the mirror use information set at the time of photography are appended to image data of each of the intraoral photographs, and in the image processing apparatus, determining whether or not it is necessary to perform at least one of horizontal reversal processing and vertical rotation processing on the image data in the image file based on the site information and the mirror use information, and performing the processing determined to be necessary.

Further, according to the present invention, provided is a non-transitory computer-readable storage medium, the storage medium storing a program that is executable by the computer, wherein the program includes program code for causing the computer to execute the image processing method comprising: acquiring image data of a plurality of intraoral photographs; determining whether or not a site information indicating a site in an oral cavity, and mirror use information indicating whether or not a mirror was used at a time of photography are appended to the image data of each of the intraoral photographs; inferring, in a case where neither of the site information and the mirror use information is appended, the site and whether or not a mirror was used based on a characteristic of each of the intraoral photographs; and storing the site information and the mirror use information by appending them to the image data of each of the intraoral photographs in a memory based on the inferred site and whether or not a mirror is used.

Further, according to the present invention, provided is a non-transitory computer-readable storage medium, the storage medium storing a program that is executable by the computer, wherein the program includes program code for causing the computer to function as an image processing apparatus comprising: an acquisition unit that acquires an image file including first image data of an intraoral photograph, site information indicating a site in an oral cavity, and mirror use information indicating whether or not a mirror was used at a time of photography; and a processor that determines whether or not it is necessary to perform at least one of horizontal reversal processing and vertical rotation processing on the first image data in the acquired image file based on the site information and the mirror use information, and performs the processing determined to be necessary.

Further, according to the present invention, provided is a non-transitory computer-readable storage medium, the storage medium storing a program that is executable by the computer, wherein the program includes program code for causing the computer to function as an image processing apparatus comprising: an acquisition unit that acquires image data of a plurality of intraoral photographs; a determination unit that determines whether or not a site information indicating a site in an oral cavity, and mirror use information indicating whether or not a mirror was used at a time of photography are appended to the image data of each of the intraoral photographs acquired by the acquisition unit; an inference unit that, in a case where neither of the site information and the mirror use information is appended, infers the site and whether or not a mirror was used based on a characteristic of each of the intraoral photographs; and a memory that stores the site information and the mirror use information by appending them to the image data of each of the intraoral photographs based on the site and whether or not a mirror is used inferred by the inference unit.

Further features of the present invention will become apparent from the following description of exemplary embodiments (with reference to the attached drawings).

### BRIEF DESCRIPTION OF THE DRAWINGS

The accompanying drawings, which are incorporated in and constitute a part of the specification, illustrate embodiments of the invention, and together with the description, serve to explain the principles of the invention.

FIG. 1 is a block diagram showing a configuration of a dental system according to an embodiment of the present invention;

FIG. 2 is a block diagram showing a hardware configuration of an image processing apparatus according to the embodiment;

FIG. 3 is a block diagram showing a schematic functional configuration of a digital still camera according to the embodiment;

FIG. 4 is a schematic rear view of the digital still camera according to the embodiment;

FIG. 5 is a flowchart showing shooting control processing in a first embodiment;

FIG. 6 is a flowchart showing image processing based on incidental information in the first embodiment;

FIG. 7 is a diagram showing an example of a user interface of the image processing apparatus according to the first embodiment;

FIGS. 8A and 8B illustrate a flowchart showing estimation processing of estimating a part of an oral cavity in a photograph and image processing according to a second embodiment;

FIGS. 9A to 9F are conceptual diagrams showing possible arrangements of photographs according to the second embodiment;

FIGS. 10A and 10B are diagrams showing a table of inference results of a condition of each tooth based on an occlusal surface view photograph in the second embodiment;

FIGS. 11A and 11B are diagrams showing a table of inference results of a condition of each tooth based on lateral view photographs assuming that no mirror was used in the second embodiment;

FIGS. 12A and 12B are diagrams showing a table of inference results of a condition of each tooth based on lateral view photographs assuming that a mirror was used in the second embodiment;

FIG. 13 is a diagram showing a table of inference patterns of placement of photographs based on the condition of each tooth in the second embodiment; and

FIG. 14 is a diagram showing an example of photographs according to a modification.

### DESCRIPTION OF THE EMBODIMENTS

Hereinafter, embodiments will be described in detail with reference to the attached drawings. Note, the following embodiments are not intended to limit the scope of the claimed invention, and limitation is not made an invention that requires a combination of all features described in the embodiments. Two or more of the multiple features described in the embodiments may be combined as appropriate. Furthermore, the same reference numerals are given to the same or similar configurations, and redundant description thereof is omitted.

### First Embodiment

### Definition of Terms

Intraoral photographs in this embodiment are visible light image data captured by the 5-sheet method in dental diagnosis. Five photographs in the five-sheet method consist of photographs of a front view, an occlusal surface view of the maxilla, an occlusal surface view of the mandible, a left lateral view, and a right lateral view. In this embodiment, the occlusal surface views of the maxilla and the mandible are collectively referred to as occlusal surface views. A combination of left and right lateral views is correctively referred to as lateral views. Further, the site in the oral cavity in the present embodiment is any one of the front, maxilla, mandible, left part, and right part.

The dental notation in this embodiment is tooth numbering system for representing the positions of teeth by numbers or alphabets in ascending order from the front to the back for the upper right teeth, the upper left teeth, the lower right teeth, and the lower left teeth. For permanent teeth, the central incisor is represented by 1 and the third molar is represented by 8. In the case of deciduous teeth, the deciduous central incisor is represented by A and the deciduous second molar is represented by E. In the present invention, the dental notation refers to a notation such as “upper right 6”. On the other hand, the type of tooth is a designation based on the shape of the tooth. From front to the back for the upper right teeth, the upper left teeth, the lower right teeth, and the lower left teeth, there are central incisor, lateral incisor, canine, (first, second) premolars, and (first, second, third) molars. In the case of deciduous teeth, they are deciduous central incisor, deciduous lateral incisor, deciduous canine, and (first and second) deciduous molars. The dental notation identifies each tooth and is described separately from the type of tooth.

Further, in this embodiment, the first to eighth teeth located on the right side of the maxilla are denoted by uR1 to uR8, and the first to eighth teeth located on the left side of the maxilla are denoted by uL1 to uL8. Also, the first to eighth teeth located on the right side of the mandible are represented by bR1 to bR8, and the first to eighth teeth located on the left side of the mandible are represented by bL1 to bL8.

The tooth condition in this embodiment indicates the health condition of the tooth, the presence or absence of dental treatment, and the like. For each tooth, at least one is selected from intact, caries, filling, crown, implant, and the like. Dental information in this embodiment is information that associates the dental notation, the condition of each tooth, and image data for each patient.

### [Prerequisites for Intraoral Photography]

For intraoral photographs taken with the five-sheet method, the state in which the appearance of the photographs is consistent with how the patient's oral cavity actually look when it is observed refers to states as follows. In maxillary occlusal surface view, the state is that the dental arch is convex. In mandible occlusal surface view, the state is that the dental arch is concave. In the front view, the state is that the direction toward the patient's nose is upward and the direction toward the patient's jaw is downward. In the left lateral view, the direction toward the patient's nose is upward and the direction toward the patient's jaw is downward, as in the front view, and the back teeth are on the right side and the front teeth are on the left side. In the right lateral view, the direction toward the patient's nose is upward and the direction toward the patient's jaw is downward, as in the front view, and the back teeth are on the left side and the front teeth are on the right side.

As a general photographing method for intraoral photography in the five-sheet method, a mirror is not used for the front view, and the patient is photographed directly. The occlusal surface is usually photographed using a mirror. This is because the back teeth cannot be photographed without using a mirror. In a case where the patient is lying down and the occlusal surface is photographed using a mirror, both maxillary and mandible dental arches are generally convex. As for a case of photographing the lateral views, there are cases where a mirror is used, and there are cases where the mouth is only widened with a mouth hook and no mirror is used. When the photograph is taken using a mirror, the view of the patient's oral cavity in the photograph is reversed from the left to right with respect to the way that the patient's oral cavity looks without using the mirror.

It should noted that, when taking a series of photographs, it is assumed that a mirror is either used or not used for the same patient, and a case where the mirror is used only for the right lateral view and not used for the left lateral view will not occur.

### [System Configuration]

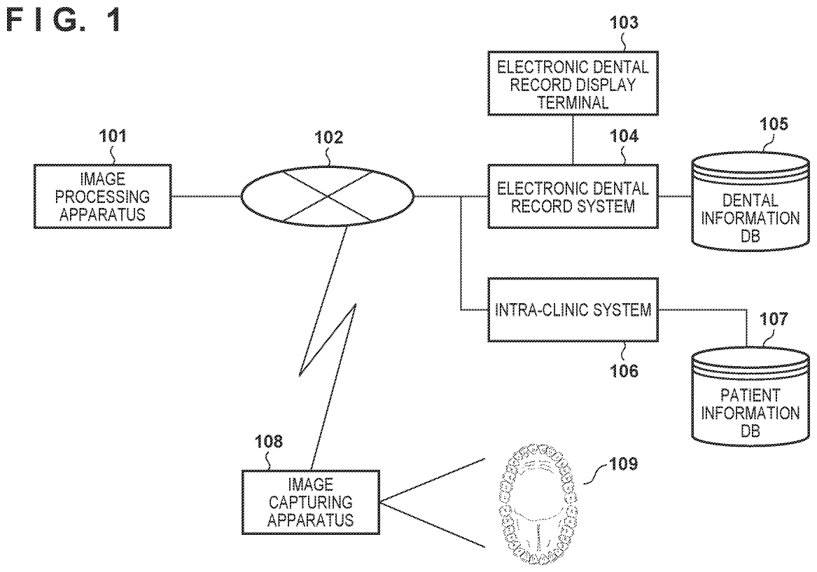

FIG. 1 is a block diagram showing the configuration of a dental system as an image capturing system of this embodiment.

An image capturing apparatus 108 takes photographs of an oral cavity 109 of a patient to be examined and generates visible light image data. In the following description, the photographs taken by the image capturing apparatus 108 will be described as visible light image data. Further, the image capturing apparatus 108 adds information of the patient such as a patient ID to captured image data and transmits the captured image data to an image processing apparatus 101, or deletes the captured image data in response to a request from the image processing apparatus 101. The image capturing apparatus 108 is also equipped with an input/output device (not shown) for confirming captured photographs and selecting information of the patient, like a commercially available digital camera.

The image processing apparatus 101 determines dental notation and condition of teeth based on information including the image data transmitted from the image capturing apparatus 108. Note that information addition processing for intraoral sites (front, maxilla, mandible, left, right) of the intraoral photographs performed by an electronic dental record display terminal 103, which will be described later, may be performed by the image processing apparatus 101 using a method for determining dental notation and condition of teeth.

The image processing apparatus 101 also communicates with the image capturing apparatus 108 and an electronic dental record system 104. Then, when the image capturing apparatus 108 is operated to turn on the power or to reconfirm the information of a patient, the information of the patient to be examined is acquired from the electronic dental record system 104, and transmitted to the image capturing apparatus 108.

The electronic dental record display terminal 103 communicates with the electronic dental record system 104, displays the electronic dental record, accepts input when the electronic dental record is created, and adds information on intraoral sites of the intraoral photographs (front, maxilla, mandible, left side, right side).

When the electronic dental record system 104 receives a request from the electronic dental record display terminal 103, it communicates with the image processing apparatus 101, an intra-clinic system 106, and a dental information database (DB) 105 to transmit and receive data necessary for creating the electronic dental record.

The dental information DB 105 communicates with the electronic dental record system 104, to store, transmit and receive the patient's dental information in association with the information of the patient such as the patient ID.

The intra-clinic system 106 communicates with a patient information DB 107 to register and acquires patient information, and communicates with the electronic dental record system 104 to transmit the patient information. The patient information DB 107 communicates with the intra-clinic system 106 to store, transmit and receive the patient information. In this way, the electronic dental record system 104 acquires the information of the patient to be examined from the patient information DB 107 via the intra-clinic system 106.

The image processing apparatus 101, the image capturing apparatus 108, the electronic dental record system 104 and the intra-clinic system 106 communicate via a network 102. Communication between the electronic dental record display terminal 103 and the electronic dental record system 104 is performed via an HDMI (registered trademark) cable or the like. Communication between the dental information DB 105 and the electronic dental record system 104 and communication between the intra-clinic system 106 and the patient information DB 107 are performed via a USB cable or the like. In the above example, the communication means are network, HDMI, and USB, but the present invention is not limited to these, and wireless communication may be used, for example.

[Computer that Operates as Image Processing Apparatus]

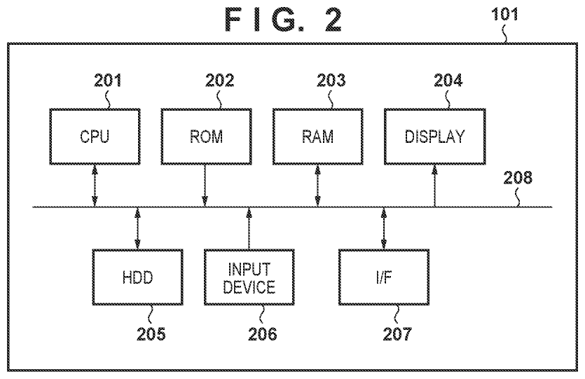

FIG. 2 is a block diagram showing a hardware configuration of the image processing apparatus 101 in this embodiment. The image processing apparatus 101 has a processor (CPU) 201 that executes programs, a Read Only Memory (ROM) 202 that stores the programs, a Random Access Memory (RAM) 203 for loading data necessary for executing the programs, and a Hard Disk Drive (HDD) 205 that stores inference data, inference results, data for generating inference data (for example, learning data), and the like. Further, The image processing apparatus 101 has an input device 206 used when registering setting information for programs, a display 204 for confirmation, an Interface (I/F) 207 to be used in communication with an external system, and a bus 208.

Each function in the image processing apparatus 101 is realized by loading a predetermined program onto hardware such as the CPU 201 and the ROM 202 and causing the CPU 201 to perform operations. It is also realized by communicating with an external system via the I/F 207, reading and writing data to/from the RAM 203 and HDD 205, and performing operations by the CPU 201.

[Digital Still Camera that Operates as an Imaging Device]

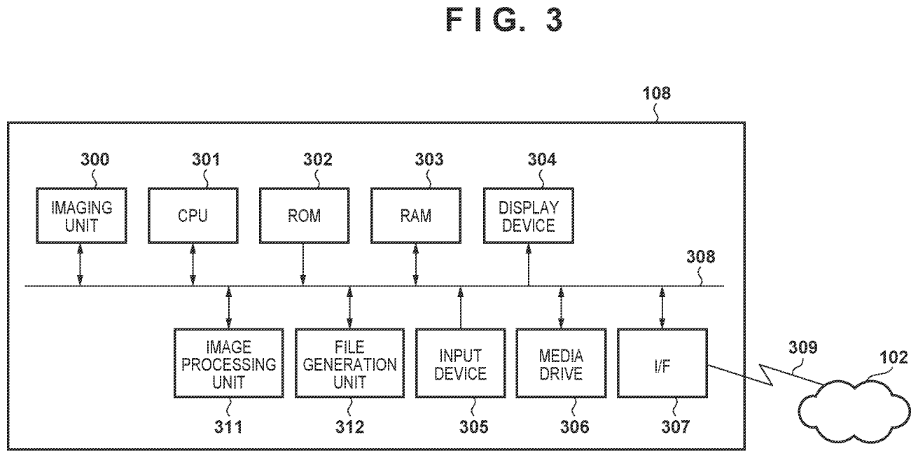

FIG. 3 is a block diagram showing a functional configuration of a digital still camera as an example of the image capturing apparatus 108 in this embodiment. By executing a predetermined control program in the digital still camera, the photographing process described below is realized, and the digital still camera functions as the image capturing apparatus 108.

An imaging unit 300 converts an incident optical image into an electrical signal using a solid-state image sensor, and performs analog-to-digital conversion on the obtained electrical signal to generate image data.

A CPU 301 controls the entire digital still camera. A ROM 302 stores operation processing procedures of the CPU 301 (for example, programs for processing when the power of the digital still camera is turned on, basic input/output processing, and the like). A RAM 303 functions as a main memory of the CPU 301, and various programs including a control program for realizing processing to be described later are loaded from the ROM 302 or the like and executed by the CPU 301. Also, the RAM 303 provides a work area when the CPU 301 executes various processes.

A display device 304 displays various contents under the control of the CPU 301. For example, it displays data stored in a storage medium (not shown).

An input device 305 is composed of one or a combination of switches, dials, touch panels, pointing by line-of-sight detection, voice recognition devices, etc. for performing various operations, and includes, for example, a release button arranged on the top of the digital still camera.

A media drive 306 accepts a detachable storage medium, and enables data to be stored in the storage medium and data stored in the storage medium to be read out.

A network interface 307 is connected to the network 102 via a communication line 309. Via this network interface 307, data is transmitted/received to/from the image processing apparatus 101, a server computer (not shown) and/or a personal computer (not shown).

A system bus 308 consists of an address bus, a data bus and a control bus, and connects the units described above.

An image processing unit 311 performs image processing on the image data output from the imaging unit 300. The CPU 301 temporarily stores the image data generated by the imaging unit 300 and the attribute information at that time in the RAM 303. Then, the image processing unit 311 performs a series of image processing so that the image data matches the human visual characteristics as needed.

Under the control of the CPU 301, a file generation unit 312 converts the image data processed by the image processing unit 311 and stored in the RAM 303 into image data in a general-purpose still image format such as a JPEG image.

### [User Interface of Image Capturing Apparatus]

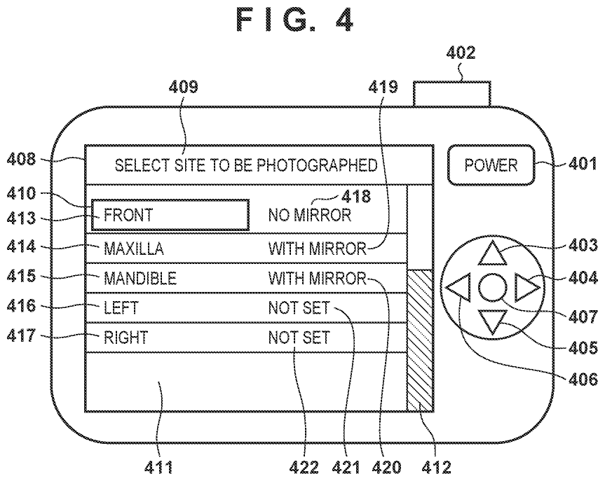

FIG. 4 is a schematic rear view of a digital still camera that functions as the image capturing apparatus 108 of this embodiment.

In FIG. 4, a power button 401 turns ON/OFF of power. When a user presses the power button 401 while the power of the digital still camera is off, the CPU 301 determines that the user has instructed to turn on the power, and turns on the power. When the user presses the power button 401 while the power is on, the CPU 301 determines that the user has instructed to turn off the power, and turns off the power.

When the power is turned on, display on a display 408 starts. Note that FIG. 4 shows an example of a screen for selecting a site of a patient to be a subject in this embodiment (hereinafter referred to as “site selection screen”).

When a release button 402 is pressed by the user, the CPU 301 determines that a still image shooting instruction has been issued.

A reference numeral 403 denotes an upward button; 404, a right button; 405, a downward button; 406, a left button; and 407, an enter button, which constitute the input device 305. When the user presses the direction buttons 403 to 406, the CPU 301 determines that the user has performed a selection switching operation, and the selected target on the display 408 is changed according to the direction of the pressed direction buttons 403 to 406. When the user presses the enter button 407, the CPU 301 determines that the user has performed a decision operation, holds the selected information in the RAM 303, and switches the state of the digital still camera.

The display 408 constitutes the display device 304 and also constitutes the input device 305 together with a touch panel laid on its surface. When the user touches an arbitrary point on the screen with his/her finger, the CPU 301 determines that there is an input instruction from the user, determines the content of the operation from the content displayed at the touched position, and executes the various processing according to the content of the operation.

Reference numerals 409 to 422 indicate the contents displayed on the display 408, and here the contents when the site selection screen is displayed are shown as described above. A region 409 displays a character string prompting the user to select one of the options. A focus frame 410 is used to notify the user of the item being selected. A selection item display area 411 indicates an area for listing the candidates for options, and here, front 413, maxilla 414, mandible 415, left 416, and right 417, which are candidates for intraoral photography are shown there. The scroll bar 412 is for changing the display area in a case where all of the candidates for options cannot be displayed in the selection item display area 411.

When the user presses the direction buttons 403 to 406, the CPU 301 determines that the user has performed a selection change operation, and moves the focus frame 410 within the selection item display area 411. Alternatively, the user can move the focus frame 410 by touching the area of the desired one of the candidates for options displayed on the display 408.

Reference numerals 418, 419, 420, 421, and 422 are menus for associating whether or not to use a mirror when photographing each part, and are hereinafter referred to as “mirror use information setting menus.” The initial value is not set, and when one of the mirror use information setting menus 418, 419, 420, 421, and 422 is selected by setting the focus frame 410, for example, a pull-down menu is displayed, and whether or not to use the mirror can be selected. Alternatively, for example, in the state in which one of the mirror use information setting menus 418, 419, 420, 421, and 422 is selected by setting the focus frame 410, the options may be changed in order by pressing the enter button 407. Thus, the selection method is not limited.

In the example shown in FIG. 4, the mirror use information setting menu 418 indicates that a mirror is not used when photographing the teeth from the front. The mirror use information setting menu 419 indicates that the mirror was used when photographing the teeth on the maxilla, and the mirror use information setting menu 420 indicates that the mirror was used when photographing the teeth on the mandible. Mirror use information setting menus 421 and 422 indicate that whether or not to use a mirror when photographing the teeth on the left and right sides has not been set.

When the enter button 407 is pressed while the focus frame 410 is at one of the mirror use information setting menus 418 to 422, or the selection is made by moving the focus frame 410 to other mirror use information setting menus 418 to 422, the CPU 301 determines the option at that time.

Information on whether or not to use a mirror may be stored in a non-volatile recording medium via the RAM 303 and media drive 306 as mirror use information in association with information on each site. The purpose of recording the mirror use information in the non-volatile recording medium is that if the site where the mirror was used is the same at the medical institution where the system is used, there is no need to set the information each time the power is turned ON/OFF. However, it is not always necessary to store the mirror use information, and it may be configured so as to be set each time photographing is performed.

### [Photographing Control Processing]

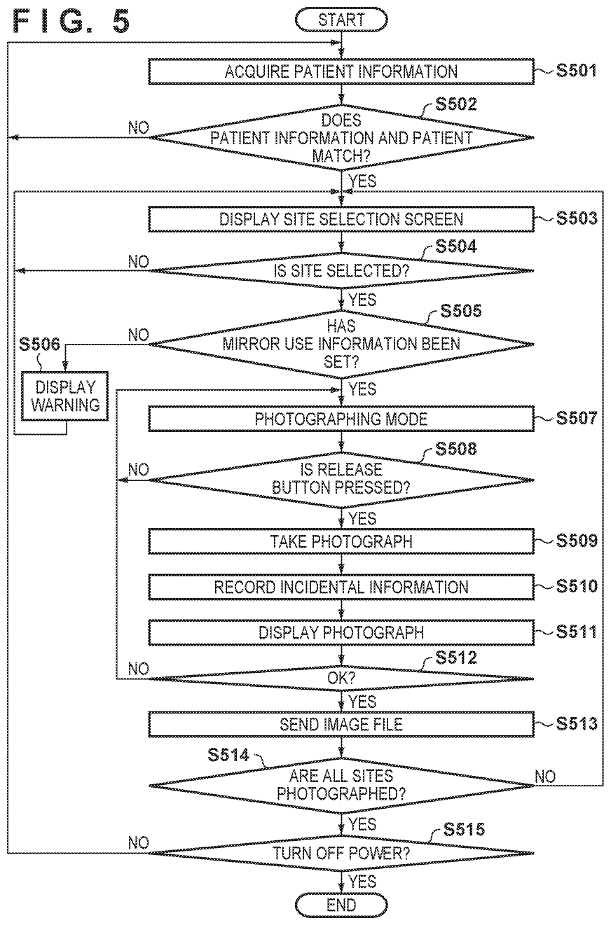

FIG. 5 is a flowchart showing photographing control processing in this embodiment. Here, a method of recording the site information and the mirror use information along with image data in the image capturing apparatus 108 will be described.

In step S501, the CPU 301 acquires patient information from the image processing apparatus 101. The patient information in this embodiment is assumed to be the patient's name, sex, and age at the time of examination.

In step S502, the CPU 301 displays the patient information received in step S501 and prompts for confirmation as to whether it matches the patient to be examined. If it is determined that the patient information does not match the patient, the process returns to step S501 and acquires the patient information from the image processing apparatus 101 again. If it is determined that the patient information matches the patient, the process proceeds to step S503. The patient information determined to match the patient is stored in the RAM 303.

In step S503, the CPU 301 displays the site selection screen shown in FIG. 4. In step S504, the CPU 301 determines whether or not a site has been selected. Whether or not a site has been selected is determined by whether or not the enter button 407 has been pressed while the focus frame 410 is located at one of the options 413 to 417. If it is determined that a site has been selected, the CPU 301 stores the selected site information in the RAM 303, and the process proceeds to step S505. At this time, the mirror use information associated with the selected site information is also stored in the RAM 303. If it is not determined that the site has been selected, the process waits for input by the user with the screen in step S503.

In step S505, the CPU 301 determines whether or not the mirror use information stored in the RAM 303 in step S504 is set. If with mirror or no mirror is set in the mirror use information setting menus, it is determined that mirror use information has been set and the process proceeds to step S507. If neither with mirror nor no mirror is set in the mirror use information setting menus, it is determined that the mirror use information has not been set and the process proceeds to step S506. In step S506, the CPU 301 displays a warning on the display 408, for example, “Please set whether or not to use a mirror”, and the process returns to step S503.

In step S507, the CPU 301 transitions to a state (shooting mode) in which the imaging unit 300 can take a photograph.

In step S508, the CPU 301 determines whether the release button 402 has been pressed. If the release button 402 has been pressed, the CPU 301 determines that a shooting instruction has been given by the user, and the process advances to step S509. If the release button 402 has not been pressed, the CPU 301 returns to step S507 assuming that the user has not yet issued a shooting instruction.

In step S509, the CPU 301 controls the imaging unit 300 to take a photograph and acquire image data.

In step S510, the CPU 301 controls the image processing unit 311 and the file generation unit 312 to convert the image data obtained in step 509 into image data in a general-purpose file format. Then, the patient information, shooting date/time information, selected site information, and mirror use information stored in the RAM 303 are recorded as incidental information in association with the image data. In the present embodiment, JPEG is used as an example of a general-purpose file format, and the incidental information is recorded as header information of the JPEG file.

In step S511, the CPU 301 displays the image captured in step S509 and an OK button on the display 408 to confirm with the user whether or not the desired photograph was taken. In step S512, the CPU 301 determines whether or not the OK button has been pressed by the user. Depression of the OK button is determined by a touch operation on the display 408 or depression of the enter button 407. If it is determined that the OK button has been pressed, the process proceeds to step S513. On the other hand, if it is determined that the OK button has not been pressed for a predetermined period, the process returns to step S507 to prompt re-taking a photograph. It should be noted that an NG button may be further displayed on the display 408, and when the NG button is pressed, the process may return to step S507.

In step S513, the CPU 301 transmits the image file generated in step S510 to the image processing apparatus 101 via the network 102. Also, the selected site information is stored in the RAM 303 as photographed site information in association with the patient information.

In step S514, the CPU 301 determines whether or not all sites to be photographed have been shot. If it is determined that all sites have been shot, the process advances to step S515. If it is determined that there is any site that has not been photographed, the process returns to step S503. In this embodiment, if image data of front, mandible, maxilla, left, and right is stored in the RAM 303 as the photographed site information for a given patient, the CPU 301 determines that all sites have been shot.

In step S515, the CPU 301 determines whether the user has pressed the power button 401 to turn off the power. If it is determined that an operation to turn off the power has been performed, the series of processes ends. If no operation to turn off the power has been performed, the process returns to step S501.

### [Image Processing Based on Incidental Information]

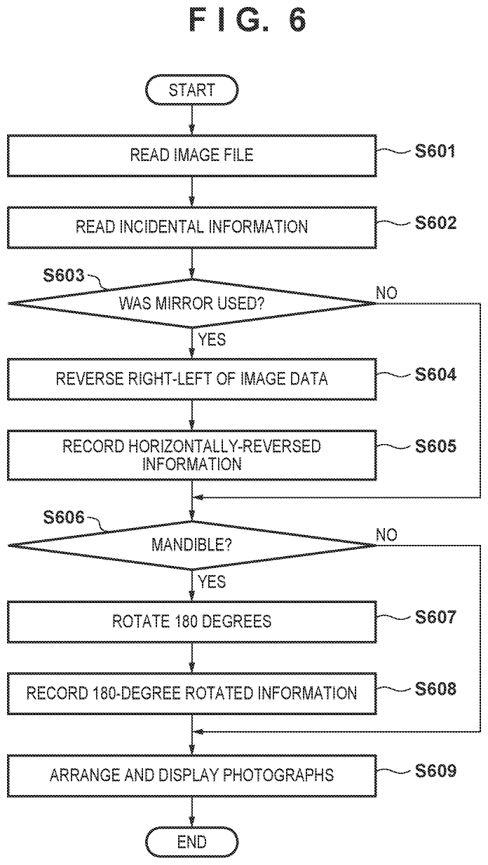

FIG. 6 is a flowchart showing image processing based on the incidental information in this embodiment. Here, a method of performing horizontal reversal processing and vertical rotation processing using the incidental information associated with image data in the image processing apparatus 101 will be described.

In step S601, the CPU 201 stores the image file received from the image capturing apparatus 108 in the RAM 203. In step S602, the CPU 201 reads the incidental information from the image file. The incidental information read at this time is the selected site information and the mirror use information associated with each image file.

In step S603, the CPU 201 uses the mirror use information to determine whether the image data of an image stored in the image file was photographed using a mirror. If it is determined that a mirror was used, the process proceeds to step S604. If it is determined that the mirror was not used, the process proceeds to step S606.

In step S604, the CPU 201 reverses the right-left of the image data in the image file. This is because, when a mirror was used, the left and right sides of the image are opposite to those seen when the subject is actually observed. In step S605, the CPU 201 records horizontally-reversed information indicating that the image has been horizontally reversed in association with the image data. In this embodiment, it is assumed that the information is recorded as header information of the image file.

In step S606, the CPU 201 determines whether the selected site information indicates mandible. If mandible, the process proceed to step S607; if not mandible, the process proceed to step S609. As described above, when the patient is laid down and the occlusal surface is photographed using a mirror, dental arches of both maxilla and mandible generally have a convex shape in the photographs. Therefore, in the case of mandible, in step S607, the CPU 201 rotates the image data in the image file by 180 degrees so that the dental arch becomes to have a concave shape. Subsequently, in step S608, the CPU 201 records 180-degree rotated information indicating that the image data has been rotated 180 degrees in association with the image data. In this embodiment, it is assumed that the information is recorded as header information of the image file.

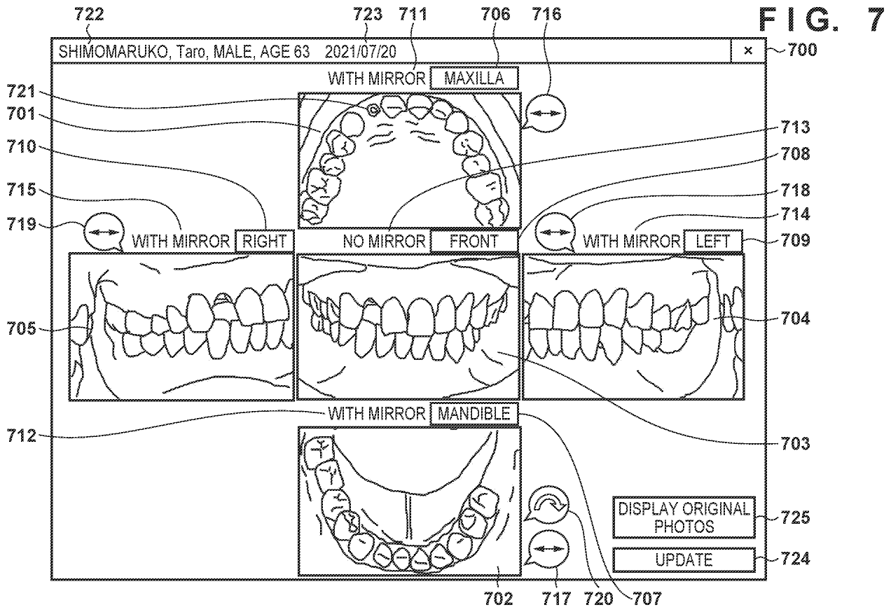

In step S609, the CPU 201 arranges and displays the five photographs on the display 204 according to the selected site information. An example of the user interface at this time will be described with reference to FIG. 7. When the display of the photographs is completed, the processing is ended.

### [User Interface of Image Processing Apparatus]

FIG. 7 is a diagram showing an example of the user interface of the image processing apparatus according to this embodiment. A method of arranging and displaying image data according to the selected site information will be described with reference to FIG. 7.

A reference numeral 700 indicates an end button, and when the user presses it, the CPU 201 determines that the user has given an end instruction, and ends the series of processes.

A reference numeral 701 indicates a maxillary occlusal surface view display area. The CPU 201 displays image data whose selected site information of the image file is maxilla in this area.

A reference numeral 702 indicates a mandible occlusal surface view display area. The CPU 201 displays image data whose selected site information of the image file is mandible in this area.

A reference numeral 703 indicates a front view display area. The CPU 201 displays image data whose selected site information of the image file is front in this area.

A reference numeral 704 indicates a left lateral view display area. The CPU 201 displays image data whose selected site information of the image file is left in this area.

A reference numeral 705 indicates a right lateral view display area. The CPU 201 displays image data whose selected site information of the image file is right in this area.

Reference numerals 706, 707, 708, 709, and 710 indicate selected site information display areas, and the CPU 201 displays the selected site corresponding to each image. If the selected site information set in the image capturing apparatus 108 is incorrect, a pop-up menu may be displayed when any of the site information display areas is clicked with an input device such as a mouse so that the site may be changed.

Reference numerals 711, 712, 713, 714, and 715 indicate mirror use information display areas. The CPU 201 displays the mirror use information corresponding to each image. If the mirror use information set in the image capturing apparatus 108 is incorrect, a pop-up menu may be displayed when any of the mirror use information display areas is clicked with an input device such as a mouse so that the mirror use information may be changed.

Reference numerals 716, 717, 718, and 719 indicate horizontal reversal icons. The CPU 201 displays the icons 716, 717, 718, and 719 only for the images to which the horizontally-reversed information is attached.

A reference numeral 720 indicates a 180-degree rotation icon. The CPU 201 displays the icon 720 only for the image to which the 180-degree rotated information is attached.

A mark 721 indicates that there is a tooth stump. If there is any information obtained by analyzing the photograph and estimating the condition of the teeth, displaying the information superimposed on the photograph will assist the diagnosis. Regarding the information related to the coordinate position of the photograph as described above, when the image is horizontally reversed and/or rotated by 180 degrees, the coordinate position is also horizontally reversed and/or rotated by 180 degrees.

A patient information display area 722 displays the patient's name, sex, and age at the time of examination. In this embodiment, as for the photographs displayed in the display areas 701 to 705, the photographs are displayed on the conditions that the photographs are of the same patient and that the examination dates match the date of photography. If the conditions are not met, the photographs are not displayed on the same screen.

A reference numeral 723 indicates an examination date and time display area. In this embodiment, it is assumed that the correct date and time are set in the image capturing apparatus, and that the date and time of photography match the date and time of examination.

A reference numeral 724 indicates an update button. If there is an operation by the user to change information in the selected site information display area or the mirror use information display area, the CPU 201 assumes, in response to the pressing operation of the update button 724, that the user has performed a display update operation, and re-executes the series of processes in FIG. 6 according to the updated information. Further, in a case where an original photograph display button 725 is pressed to display a photograph before being horizontally reversed or rotated, the series of processes in FIG. 6 is re-executed when the update button 724 is pressed.

If the user presses the original photograph display button 725, the CPU 201 assumes that the user has performed an original photograph display operation, and displays the photographs immediately after shooting and before being horizontally reversed or rotated in respective image areas. Further, if the original photograph display button 725 is pressed, an edited photograph display button may be displayed instead. By switching the display to the edited photograph display button, it is possible to easily change the displayed photographs from the photographs immediately after shooting to the edited photographs.

In this embodiment, the image data in the image file is updated in steps S604 and S607 according to the horizontal reversal or 180-degree rotation. However, the present invention is not limited to this, and the image data itself in the image file may not be edited, and only a horizontal reversal flag and/or a 180-degree rotation flag may be recorded in association with the image data. In such case, the image data read into the RAM 203 may be horizontally reversed and/or rotated according to the flags when the user interface shown in FIG. 7 is displayed. By doing so, it is possible to shorten the processing time required for writing to the image file caused by horizontal reversal and 180-degree rotation. In that case, the horizontal reversal icons 716, 717, 718, 719 and the 180-degree rotation icon 720 are displayed depending on whether or not horizontal reversal and/or 180-degree rotation is performed at the time of displaying the image data. Furthermore, when the original photograph display button 725 is pressed, horizontal reversal or rotation processing according to the flag is not performed at the time of displaying the image data.

As described above, according to the first embodiment, the image data is horizontally inverted and/or rotated by 180 degrees based on the selected site information and the mirror use information selected at the time of photography. This makes it possible to display and record photographs after being arranged so that the photographs match how teeth of the patient look when they are actually observed, enabling medical personnel to make diagnoses using appropriate photographs.

In addition, by displaying the photographs associated with the patient and the same examination date as a set, it is possible to prevent errors in diagnosis and recording due to discrepancies between the patient and the photographs.

In addition, by viewing intraoral photographs while confirming the selected site information and mirror use information selected at the time of photography, if an error is noticed, it can be corrected. Then, by updating the display based on the corrected information, the error at the time of photography can be corrected and the corrected information can be recorded.

In addition, the information recorded in association with the coordinates of the photographs are horizontally reversed and/or rotated together with the photographs, so that medical personnel can appropriately utilize the information that can assist diagnosis.

In addition, whether the photograph is unedited or has been edited by a reversal and/or rotation operation, for example, can be recognized, so if it is necessary to check the photograph immediately after shooting, it is possible to check original image data of the photograph before undergone the reversal and/or rotation operation by pressing the original photograph display button.

In addition, by storing the mirror use information at the time of taking a photograph of each site, it is not necessary to set the mirror use information each time a photograph is taken in a medical institution where intraoral photography is performed in the same procedure.

### Second Embodiment

Next, a second embodiment of the present invention will be described. In the second embodiment, a case where the image processing apparatus 101 performs horizontal reversal and/or vertical rotation by inferring dental notation, tooth type, tooth condition, and site in a case where image data photographed and stored for each patient is not stored in association with the site information and the mirror use information will be described. Note that the apparatus configuration of the dental system in the second embodiment is the same as that described with reference to FIGS. 1 to 3 in the first embodiment, so the description thereof will be omitted.

In addition, in this embodiment, machine learning is used to infer dental notation, tooth type, tooth condition, and site. It should be noted that any image processing, such as pattern matching processing, discrimination processing using edge detection, gradation, or color information, and so forth, may be used as long as similar processing results can be obtained.

In addition, the rough classification of sites in this embodiment is the front view, occlusal surface views, lateral views, and others. Also, in this embodiment, photographs stored for each patient in the HDD 205 of the image processing apparatus 101 will be processed. Photographs transmitted from an external device, such as photographs captured by the image capturing apparatus 108, are stored in the HDD 205.

### [Image Processing Based on Inference Results]

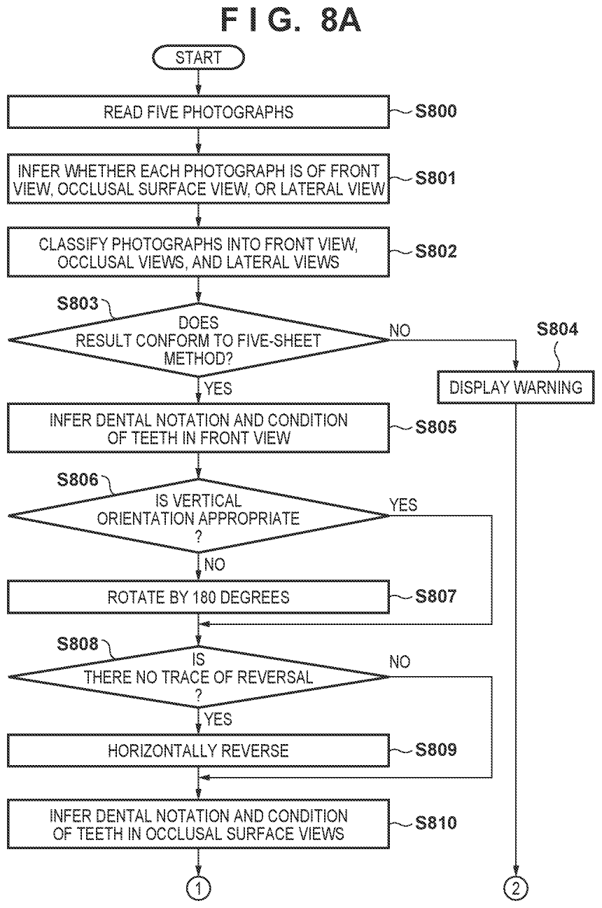

FIGS. 8A and 8B illustrate a flowchart showing inference processing of the site of a photograph and image processing in this embodiment.

First, in step S800, the CPU 201 reads image files of five photographs taken by the five-sheet method and stored for each patient from the HDD 205 to the RAM 203. Next, in step S801, the CPU 201 infers whether each of the photographs of the five image files read in step S800 is of a front view, occlusal view, lateral view, or other.

In step S802, the CPU 201 classifies the five photographs based on the inference results. If the photographs were properly taken with the five-sheet method, one photograph would be classified as a front view, two photographs would be classified as occlusal views, and two photographs would be classified as lateral views. Then, in step S803, if the results do not conform to the five-sheet method, the CPU 201 determines that the input photographs are not appropriate, displays a warning in step S804, and terminates the processing. If the results conform to the five-sheet method, the process proceeds to step S805.

In step S805, the CPU 201 records “front view” as site information of the photograph classified as a front view in association with the image data, and also infers dental notation, tooth type, and condition.

In step S806, the CPU 201 determines whether the vertical orientations of the photographs are appropriate. Whether or not the vertical orientations are appropriate is determined by whether or not the maxillary teeth exist in the upper part of the photograph and the mandible teeth exist in the lower part of the photograph. If it is determined that the vertical orientations are appropriate, the process proceeds to step S808. If it is determined that the vertical orientations are inappropriate, the process advances to step S807. For example, when the patient is laid down and photographed, if the patient is photographed from the front where the photographer is at the patient's head, an upside-down photographs will be taken.

In step S807, the CPU 201 rotates the image data in the RAM 203 determined to be vertically inappropriate in step S806 by 180 degrees. Then, a 180-degree rotation flag is stored in association with the image data.

Next, in step S808, the CPU 201 determines whether or not there is a trace of reversal in each of the two images classified as occlusal surface views. Whether or not there is a trace of reversal is determined by whether or not the shooting date and time recorded in the header of the image file match the update date and time of the file. If they do not match, it is determined that there is a trace of reversal. If they match, it is determined that there is no trace of reversal. If there is a trace of reversal, the process proceeds to step S809. If there is no trace of reversal, the process proceeds to step S810.

In step S809, the CPU 201 horizontally reverses the image data in the RAM 203 that corresponds to the photograph or photographs that do not have a trace of reversal of the occlusal surface among the two photographs classified as occlusal surface views. Then, a horizontal reversal flag is stored in association with the image data. This is because, as described above, the photographs of the occlusal surfaces are generally taken using a mirror.

In step S810, the CPU 201 infers dental notation and tooth condition for the two photographs classified as occlusal surface views. In this embodiment, object detection in machine learning is used to infer the dental notation and tooth condition use, and an inference result is obtained as information of a rectangular portion corresponding to the coordinates on each photograph. As a result of the inference, the CPU 201 determines the photograph in which the maxillary teeth appear as the photograph of the maxillary occlusal surface view, and the photograph in which the mandible teeth appear as the photograph of the mandible occlusal surface view, and records the site information in association with the photographs. In addition, since the photographs of the occlusal surface views are always taken using a mirror, the mirror use information is also associated and recorded.

In step S811, the CPU 201 determines whether or not the dental arch has a convex shape in spite that the mandible teeth appear in step S810. Whether or not the dental arch is convex is determined from the alignment of the rectangular images detected as teeth. If it is determined that the dental arch is convex despite mandible teeth, the process proceeds to step S812. If it is determined that the dental arch is concave and mandible teeth appear, the process proceeds to step S813.

In step S812, the CPU 201 rotates the image data in the RAM 203 in which the dental arch is convex in spite that the occlusal surface is of mandible by 180 degrees. Then, a 180-degree rotation flag is stored in association with the image data.

Next, in step S813, the CPU 201 infers dental notation and tooth condition for each of the two photographs classified as lateral views.

Then, in step S814, the CPU 201 assumes that the two photographs classified as lateral views are taken without using a minor, and compares the inference result of the presence or absence of tooth, the metal prosthesis, etc. with the inference result of teeth in the occlusal surface view. At this time, the site of the photograph where the back teeth are on the right side and the front teeth on the left side is assumed as left and the site of the photograph where the back teeth are on the left side and the front teeth are on the right side is assumed as right, and the inference results of the lateral views and the occlusal views are compared. Then, the comparison result is stored in the RAM 203 as a comparison result of each tooth assuming that the photographs of the lateral views are taken without using a mirror.

Next, in step S815, the CPU 201 horizontally reverses the dental notation of the teeth in the photographs classified as the lateral views, and compares the inference result of the condition of the teeth of the lateral views when it is assumed that the two photographs were taken using a mirror, and the inference result of the occlusal view. Then, the comparison result is stored in the RAM 203 as a comparison result of each tooth assuming that the photographs of the lateral views are taken using a mirror.

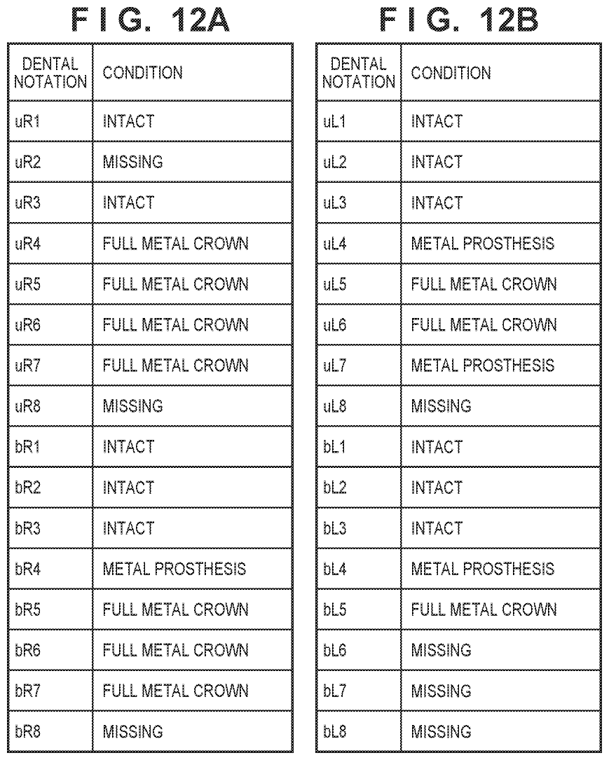

In step S816, the CPU 201 uses the comparison results of each tooth stored in steps S812 and S813 to determine for each tooth whether or not photographs were taken using a mirror. Details of the determination performed here will be described later with reference to FIG. 13.

In step S817, the CPU 201 determines whether or not the photographs were taken using a mirror by integrating the inference results for each tooth, and decides whether or not the photographs were taken using a mirror, or without using a minor, or the results contradict or are undecided. In a case where contradiction or undecided is decided, the process proceeds to step S820. In a case where neither contradiction nor undecided is decided, the process proceeds to step S818.

In step S818, the CPU 201 determines which of the photographs of the lateral views is of right or of left based on the result of comprehensive determination. At this time, if the photographs assumed to have been taken using a mirror are selected in step S815, the image data of the two lateral views in the RAM 203 are horizontally reversed. Then, a horizontal reversal flag is stored in association with the image data, and the site information and the minor use information are recorded. If the photographs assumed to have been taken without using a minor are selected, the image data of the two lateral views are not horizontally reversed, and the site information and the mirror use information indicating taken without a mirror are recorded.

In step S819, the CPU 201 appropriately performs the processing in FIG. 6 for the front view, maxillary occlusal surface view, mandible occlusal surface view, left lateral view, and right lateral view classified in the above process, and displays the processed photographs by using the interface shown in FIG. 7. Then, the series of processes ends.

On the other hand, in step S820, the CPU 201 determines whether or not there is a contradiction. If there is a contradiction, the process proceeds to step S821. If there is no contradiction, the process proceeds to step S822.

In step S822, since the CPU 201 cannot determine whether a mirror was used or not, so it issues a notification stating that “no characteristic difference between left and right teeth is found, so correction will not be made, and two photographs of lateral views are displayed with the photograph in which the back teeth are located on the right side and the front teeth are located on the left side being the left lateral view, and the photograph in which the back teeth are located on the left side and the front teeth are located on the right side being the right lateral view. Further, it is assumed that mirror is not used. Then, the site information and the mirror information are recorded in association with the image data. After that, the process proceeds to step S819.

On the other hand, in step S821, since there is no consistent combination, the CPU 201 notifies that “other patient's photographs may be mixed” and terminates the series of processes.

### [Method of Inferring the Site of the Photograph]

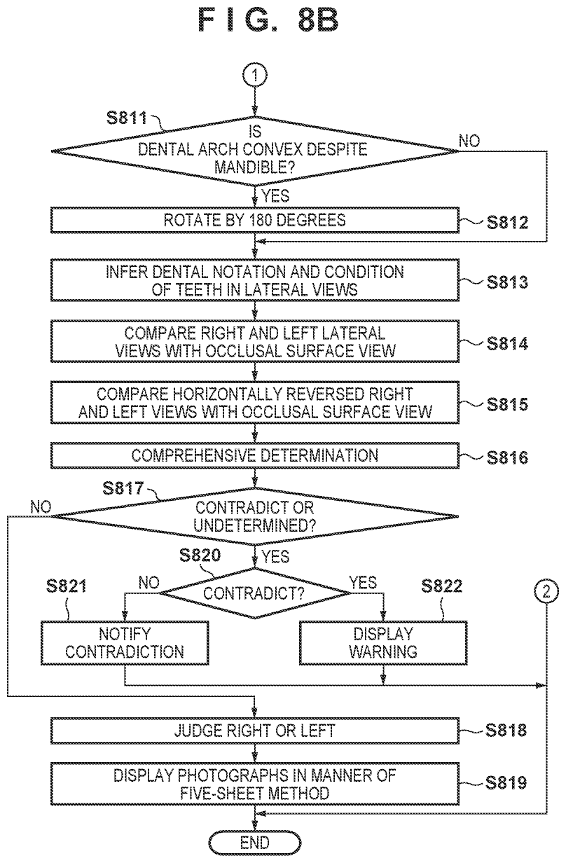

FIGS. 9A to 9F are conceptual diagrams showing examples of inferred classification results of photographs. FIG. 9A shows a photograph inferred to be of the maxilla, and FIG. 9B shows a photograph inferred to be of the mandible.

FIGS. 9C and 9D show photographs inferred to be of lateral views assuming no use of a mirror, and specifically, FIG. 9C shows a photograph inferred to a right lateral view and FIG. 9D shows a photograph inferred to be of a left lateral view. If a mirror is not used, the back teeth appear on the left and the front teeth appear on the right, so the photograph shown in FIG. 9C is assumed to be of a right lateral view. Further, if a mirror is not used, the front teeth appear on the left and the back teeth appear on the right, so the photograph shown in FIG. 9D is assumed to be of a left lateral view.

FIGS. 9E and 9F show photographs inferred to be of lateral views assuming that a mirror was used, and specifically, FIG. 9E shows the photograph inferred to be of a right lateral view, which is a horizontally-reversed photograph of the photograph in FIG. 9D. If a mirror was used, the back teeth appear on the left and the front teeth appear on the right, so the photograph shown in FIG. 9E is assumed to be of a right lateral view. FIG. 9F shows the photograph inferred to be of a left lateral view, which is a horizontally-reversed photograph of the photograph in FIG. 9C. If a mirror was used, the front teeth appear on the left and the back teeth appear on the right, so the photograph shown in FIG. 9F is assumed to be of a left lateral view.

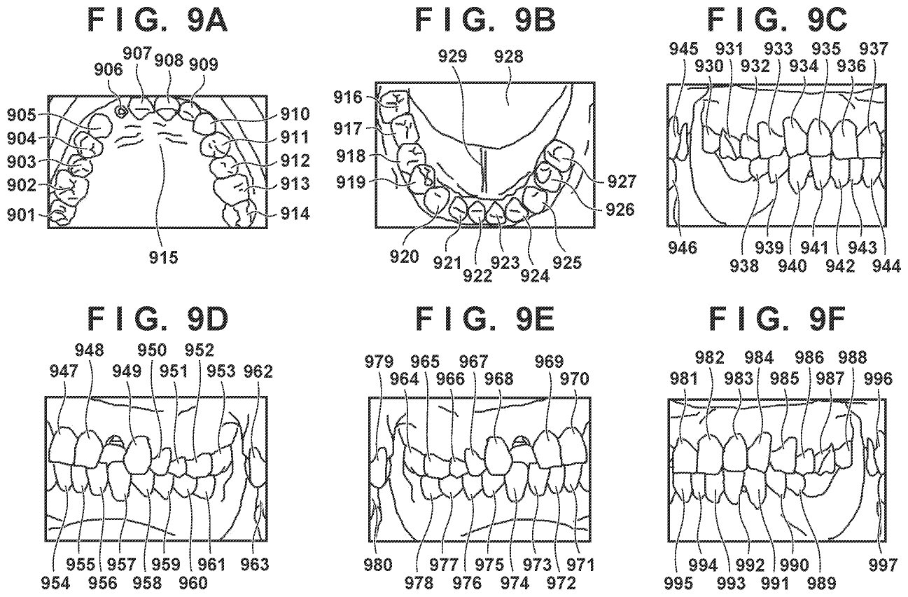

FIGS. 10A and 10B, FIGS. 11A and 11B, and FIGS. 12A and 12B are tables showing condition of teeth by associating inference results of sites with dental notations. It should be noted that the condition of teeth are associated in a case where regions of the coordinates on the photograph that results from the inference coincides with the regions of the coordinates on the photograph that result from the inference of the tooth corresponding to the dental notation.

FIG. 10A is a table showing an example of inference results of the condition of teeth 901 to 914 in the photograph of the maxilla shown in FIG. 9A. A tooth 907 in the photograph shown in FIG. 9A corresponds to the dental notation uR1 in FIG. 10A, and a tooth 901 corresponds to the dental notation uR7. A tooth 908 corresponds to the dental notation uL1, and a tooth 914 corresponds to the dental notation uL7. Reference numeral 915 indicates a characteristic portion seen only in the maxilla called palatal folds in the oral cavity.

FIG. 10B is a table showing an example of inference results of the condition of teeth 916 to 927 in the photograph of the mandible shown in FIG. 9B. A tooth 922 in the photograph shown in FIG. 9B corresponds to the dental notation bR1 in FIG. 10B, and a tooth 916 corresponds to the dental notation bR7. A tooth 923 corresponds to the dental notation bL1 and a tooth 927 corresponds to the dental notation bL5. Reference numeral 928 indicates the tongue. Reference numeral 929 indicates the tongue frenulum, a characteristic portion found only in the mandible.

As shown in FIGS. 10A and 10B, dental notations and inference results are stored. In this embodiment, if a tooth corresponding to a dental notation cannot be detected from the photograph by inference, it is treated as missing.

For example, in FIG. 10A, the condition of a tooth 907 of the dental notation uR1 is intact, and the condition of a tooth 906 of the dental notation uR2 is missing. The dental notations uR3-uR7 correspond to teeth 905-901, and the dental notations uL1-uL7 correspond to teeth 908-914. Since teeth corresponding to the dental notation uR8 and the dental notation uL8 cannot be detected, they are treated as missing. The same applies to FIG. 10B.

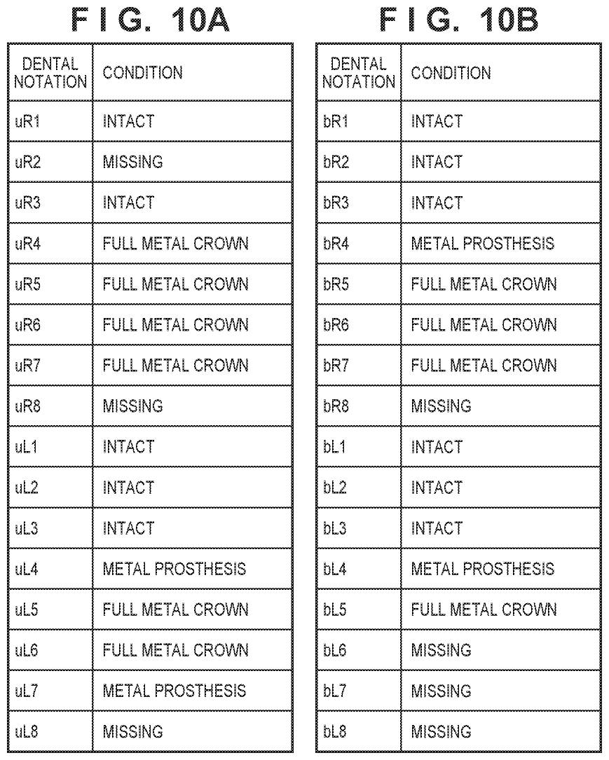

FIG. 11A is a table showing an example of inference results of the condition of teeth 930 to 944 in the photograph of a right lateral view shown in FIG. 9C assuming that no mirror was used. The tooth 936 in the photograph in FIG. 9C corresponds to the dental notation uR1 in FIG. 11A, and the tooth 930 corresponds to the dental notation uR7. Also, the tooth 942 corresponds to the dental notation bR1, and the tooth 938 corresponds to the dental notation bR5. Since inference is performed assuming that FIG. 9C shows the photograph of a right lateral view, the resulting dental notations in FIG. 11A are all of the teeth appearing on the right side of the patient. That is, the tooth 937 corresponds to the dental notation uL1, the tooth 944 corresponds to the dental notation bL2, and the tooth 943 corresponds to the tooth located at the left of a tooth of dental notation bL1, and therefore not shown in the table of FIG. 11A. Further, teeth 945 and 946 are teeth appearing outside the dentition, and are sometimes included in the photograph of the subject when a mirror was used.

The example shown in FIG. 11A, for example, indicates that the tooth 936 of the dental notation uR1 is intact. Since teeth corresponding to the dental notations uR8, bR6, bR7, and bR8 cannot be detected, they are treated as missing.

FIG. 11B is a table showing an example of the inference results of the condition of teeth 947 to 961 in the photograph of a left lateral view shown in FIG. 9D assuming that no mirror was used. The tooth 947 in the photograph in FIG. 9D corresponds to the dental notation uR1 in FIG. 11B, the tooth 948 corresponds to the dental notation uL1, and the tooth 953 corresponds to the dental notation uL7. The tooth 954 corresponds to the dental notation bR1, the tooth 955 corresponds to the dental notation bL1, and the tooth 961 corresponds to the dental notation bL7. Since inference is performed assuming that FIG. 9D shows the photograph of a left lateral view, the resulting dental notations in FIG. 11B are all of the teeth appearing on the left side of the patient. Note that teeth 962 and 963 are teeth appearing outside the dentition, and are sometimes included in the photograph of the subject when using a mirror.

The example shown in FIG. 11B, for example, indicates that the tooth 948 of the dental notation uL1 is intact. Since teeth corresponding to the dental notations uL2, uL8, and bL8 cannot be detected, they are treated as missing.

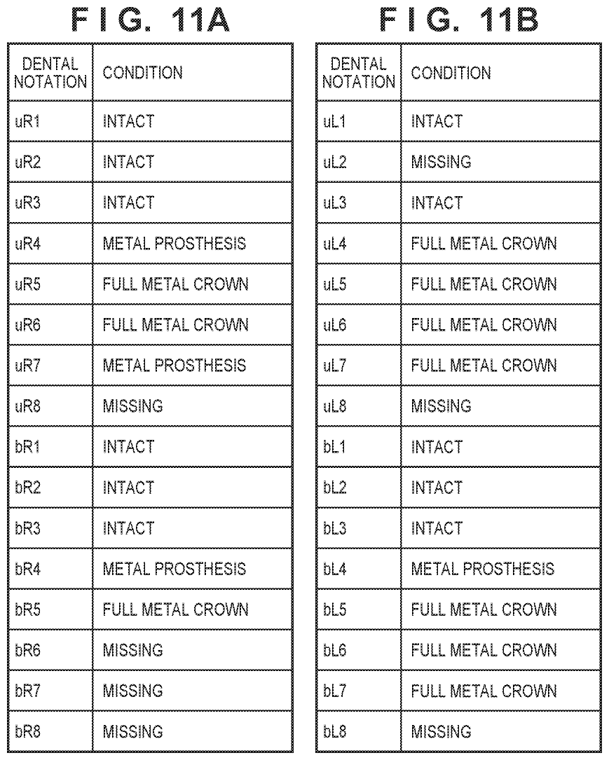

FIG. 12A is a table showing an example of inference results of the condition of teeth 964 to 978 in the photograph of a right lateral view shown in FIG. 9E assuming that a mirror was used, and L (left) of the dental notations of the inference results shown in FIG. 11B are changed to R (right). The tooth 970 in the photograph in FIG. 9E corresponds to the dental notation uL1 in FIG. 12A, the tooth 969 corresponds to the dental notation uR1, and the tooth 964 corresponds to the dental notation uR7. Further, the tooth 971 corresponds to the dental notation bL1, the tooth 972 corresponds to the dental notation bR1, and the tooth 978 corresponds to the dental notation bR7. Since inference is performed assuming that FIG. 9E shows the photograph of a right lateral view, the resulting dental notations in FIG. 12A are all of the teeth appearing on the right side of the patient. Teeth 979 and 980 are teeth appearing outside the dentition, and are sometimes included in the photograph of the subject when a mirror was used.

The example shown in FIG. 12A, for example, indicates that the tooth 969 of the dental notation uR1 is intact. Since teeth corresponding to dental notations uR2, uR8, and bR8 cannot be detected, they are treated as missing.

FIG. 12B is a table showing an example of inference results of the condition of teeth 981 to 995 in the photograph of a left lateral view shown in FIG. 9F assuming that a mirror was used, and R (right) of the dental notations of the inference results shown in FIG. 11A are changed to L (left). The tooth 981 in the photograph in FIG. 9F corresponds to the dental notation uR1 in FIG. 12B, the tooth 982 corresponds to the dental notation uL1, and the tooth 988 corresponds to the dental notation uL7. Further, the tooth 995 corresponds to the dental notation bR2, the tooth 994 corresponds to the dental notation bR1, the tooth 993 corresponds to the dental notation bL1, and the tooth 989 corresponds to the dental notation bL5. Since inference is performed assuming that FIG. 9F shows the photograph of a left lateral view, the resulting dental notations in FIG. 12B are all of the teeth appearing on the left side of the patient. Note that teeth 996 and 997 are teeth appearing outside the dentition, and are sometimes included in the photograph of the subject when a mirror was used.

The example shown in FIG. 12B, for example, indicates that the tooth 982 of the dental notation uL1 is intact. Since teeth corresponding to the dental notations uL8, bL6, bL7, and bL8 cannot be detected, they are treated as defective.

### [Description of Comprehensive Determination]

Next, how to determine whether or not photographs of lateral views are obtained using a mirror based on the above-described inference of the occlusal surface and lateral views will be explained.

FIG. 13 is a table showing the matching state of the conditions of teeth in the occlusal surface and in the lateral views and the patterns of the results inferred from the matching state. Referring to this table, for each of the teeth labelled with the dental notations in FIGS. 10A to 12B, it is determined whether each lateral view is the right lateral view or the left lateral view. As shown in FIG. 13, the combination can be divided into 11 patterns in total, regardless of matching/mismatching (indicated by *), and the matching state of the condition of tooth of each dental notation corresponds to one of the 11 patterns. The determination method in step S816 of FIG. 8B will be described below with reference to FIG. 13.

In FIG. 13, “photograph 1” and “photograph 2” indicate photographs of the lateral surface. If photograph 1 is of right lateral view without reversing the photograph, “assumed right” of photograph 1 corresponds to the photograph assuming that no mirror was used (e.g., FIG. 9C). Also, “assumed left” of photograph 1 corresponds to the photograph assuming that a mirror was used (e.g., FIG. 9F). Similarly, if photograph 2 is of left lateral view without reversing the photograph, “assuming right” of photograph 2 corresponds to the photograph assuming that a mirror was used (e.g., FIG. 9E), and “assumed left” of photograph 2 corresponds to the photograph assuming that no mirror was used (e.g., FIG. 9D).

Then, the conditions of the teeth in photographs 1 and 2 with “assumed right” and “assumed left” were compared with the conditions of teeth in the occlusal surface views of the same dental notation, thereby whether photographs 1 and 2 is of right view or left view is estimated for each tooth.

∘ indicates that the conditions of teeth of the same dental notation in the occlusal surface views are consistent with those of photographs 1 and 2, and x indicates inconsistent. Further, * does not indicate consistent or inconsistent (both consistent and inconsistent are acceptable).

For example, the condition of tooth of dental notation uR1 in FIG. 11A that shows the inference result when photograph 1 is assumed to be of right lateral view (for example, FIG. 9C) is “intact”, and the condition of the corresponding tooth of dental notation uR1 in FIG. 10A that shows the occlusal surface view is also “intact”, and thus the conditions are consistent (∘). In addition, the condition of tooth of dental notation uL1 in FIG. 12B that shows the inference result when photograph 1 is assumed to be of left lateral view (for example, FIG. 9F) is “intact”, and the condition of the corresponding tooth of dental notation uL1 in FIG. 10A that shows the occlusal surface view is also “intact”, and thus the conditions are consistent (∘). Also, the condition of tooth of dental notation uR1 in FIG. 12A that shows the inference result when photograph 2 is assumed to be of right lateral view (for example, FIG. 9E) is “intact”, and the condition of the corresponding tooth of dental notation uR1 in FIG. 10A that shows the occlusal surface view is also “intact”, and thus the conditions are consistent (∘). Further, the condition of tooth of dental notation uL1 in FIG. 11B that shows the inference result when photograph 2 is assumed to be of left lateral view (for example, FIG. 9D) is “intact”, and the condition of corresponding tooth of dental notation uL1 in FIG. 10A that shows the occlusal surface view is also “intact”, and thus the conditions are consistent (∘). In this case, the pattern 1 in FIG. 13 is applied, and determination as to which of photographs 1 and 2 is of right lateral view or left lateral view is undecided.

With the similar judgement, the condition of tooth of dental notation uR7 in FIG. 11A that shows the inference result when photograph 1 is assumed to be of right lateral view is “metal prosthesis”, but the condition of the corresponding tooth of dental notation uR7 in FIG. 10A that shows the occlusal surface view is “full metal crown”, and thus the conditions are inconsistent (x). In addition, the condition of teeth of dental notation uL7 in FIG. 12B that shows the inference result when photograph 1 is assumed to be of left lateral view is “metal prosthesis”, and the condition of the corresponding tooth of dental notation uL7 in FIG. 10A that shows the occlusal surface view is also “metal prosthesis”, and thus the conditions are consistent (∘). Also, the condition of tooth of dental notation uR7 in FIG. 12A that shows the inference result when photograph 2 is assumed to be of right lateral view is “full metal crown”, and the condition of the corresponding tooth of dental notation uR7 in FIG. 10A that shows the occlusal surface view is also “full metal crown”, and thus the conditions are consistent (∘). However, the condition of tooth of dental notation uL7 in FIG. 11B that shows the inference result when photograph 2 is assumed to be of left lateral view is “full metal crown”, but the condition of tooth of dental notation uL1 in FIG. 10A that shows the occlusal surface view is “metal prosthesis”, and thus the conditions are inconsistent (x). In this case, the pattern 7 in FIG. 13 is applied, and photograph 1 is determined to be of right lateral view and photograph 2 is determined to be of left lateral view.

In this way, similar determinations are made for all teeth using the table in FIG. 13, and it is determined whether photograph 1 and photograph 2 correspond to “undetermined”, “right”, “left”, or “contradict” shown in the determination column of FIG. 13 based on the condition of each tooth of each dental notation in photographs 1 and 2 and the condition of corresponding tooth in the occlusal surface view. Then, by integrating the obtained determination results, it is judged whether each of photograph 1 and photograph 2 is of right lateral view or of left lateral view. Specifically, the number of times photograph 1 is determined to be of right lateral view (or left lateral view) is compared with the number of times photograph 2 is determined to be of right lateral view (or left lateral view), and one of photograph 1 and photograph 2 of which the number of times determined to be of right lateral view (left lateral) is greater is determined to be of right lateral view (or left lateral view), and the other photograph is determined to be of left lateral view (or right lateral view).

However, if there are many “undetermined” or “contradict”, or if, for example, the number of times photograph 1 is determined to be of right lateral view and the number of times photograph 1 determined to be of left lateral view are the same or close to each other, it is not possible to judge which of the photographs is of right lateral view or left lateral view (NO in step S817). In such a case, in step S820, it is determined whether the determination results are inconsistent or whether the judgement cannot be made because there are many pending determinations, the process proceeds to step S822, and if not, the process proceeds to step S821.

In this embodiment, it is described that the image data in the RAM 203 is reversed or rotated in steps S807, S809, and S812 and stored in association with a flag, however, the image data in the image file may be rewritten after reversing and rotating the image data. The display process by referring to the flag or the image file updated itself is the same as the method described in the first embodiment.

As described above, according to the second embodiment, even if the photographs are stored without being associated with site information and mirror use information, it is possible to infer the site shown by each of the photographs with high precision, and perform the horizontal reversal processing and the vertical rotation processing.

### <Modification>

In the above-described second embodiment, the site shown by each photograph is inferred by inferring the condition of each tooth and analyzing the inference result. On the other hand, in this modification, an example of another inference method will be described.

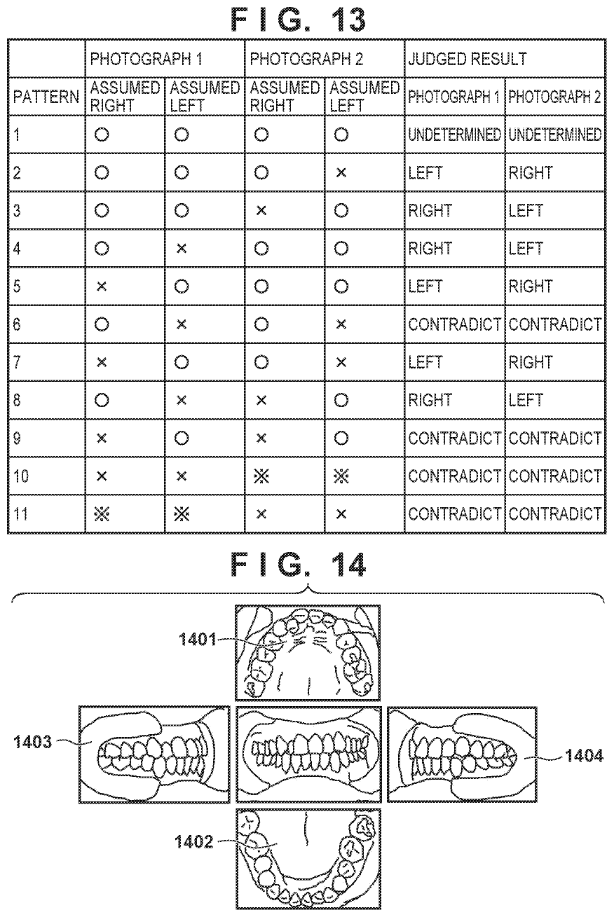

FIG. 14 is a conceptual diagram showing detection results of characteristic non-tooth tissues and instruments. Reference numeral 1401 indicates a palatal fold, reference numeral 1402 indicates a tongue, and reference numerals 1403 and 1404 indicate corner hooks.

In the second embodiment, whether a photograph is of the maxilla or mandible is determined from the inference result of teeth in step S810, but the photograph can be determined to be of the maxilla if the palatal fold 1401 is detected. Also, a photograph can be determined to be of the mandible if the tongue 1402 or the tongue frenulum 929 shown in FIG. 9B is detected.

In addition, in step S813, the conditions of teeth in the photographs of the lateral views were inferred, and with the comprehensive determination in step S816, it is judged whether the photograph is of right lateral view or left lateral view. However, if there are the corner hooks 1403 and 1404 in the photographs, the mirror usage information indicating that a mirror was not used may be recorded. Then, if the shooting date and time of the image file and the updated date and time of the file match, whether each photograph is of right lateral view or the left lateral view may be determined according to the positions of the front teeth and the back teeth without horizontally reversing the image data.

Further, if there are teeth 945, 946, 979, and 980 appearing outside the dentition as shown in FIGS. 9A to 9F, the mirror usage information indicating that a mirror was used may be recorded. Then, if the shooting date and time of the image file and the updated date and time of the file match, whether each photograph is of right lateral view or the left lateral view may be determined according to the positions of the front teeth and the back teeth after horizontally reversing the image data.

In addition, in the second embodiment, an example was described in which the dental notation and the conditions of the teeth in the occlusal surface view and those in the lateral views were closely compared. If there is a reliable premise that the set of intraoral images are of a single patient, judgement may be performed based on existence/absence of tooth, a characteristic tooth such as a full crown, and the like. Further, judgement may be performed based on the types of teeth that are less strict than dental notation, such as lateral incisors, canines, and molars. As a result, even if the accuracy of the inference is not so high, it is possible to perform horizontal reversal or 180-degree rotation of photographs, and the photographs may be arranged and displayed so as to match how teeth of the patient look when they are actually observed.

As described above, according to this modification, even if the site information and mirror use information were not recorded at the time of photography, sites shown in photographs can be inferred based on the types and conditions of teeth, characteristic parts in the oral cavity, detection results of instruments, and so forth.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-206263, filed Dec. 20, 2021 which is hereby incorporated by reference herein in its entirety.

What is claimed is:

1. An image processing apparatus comprising one or more processors and/or circuitry which functions as:
an acquisition unit that acquires an image file including first image data of an intraoral photograph, site information indicating a site in an oral cavity, and mirror use information indicating whether or not a mirror was used at a time of photography; and
a processor that determines whether or not it is necessary to perform at least one of horizontal reversal processing and vertical rotation processing on the first image data in the acquired image file based on the site information and the mirror use information, and performs the processing determined to be necessary.

2. The image processing apparatus according to claim 1, wherein the site information appended to the first image data of the intraoral photograph indicates one of front, maxilla, mandible, right, and left in a five-sheet method.

3. The image processing apparatus according to claim 2, wherein the processor performs the vertical rotation processing on the first image data in a case where the site information indicates the mandible.

4. The image processing apparatus according to claim 1, wherein the processor performs the horizontal reversal processing on the first image data in a case where the mirror use information indicates that a mirror was used.

5. The image processing apparatus according to claim 1, wherein, in a case where the first image data is processed by the processor, second image data obtained by processing the first image data is recorded in the image file.

6. The image processing apparatus according to claim 1, wherein, in a case where the first image data is processed by the processor, a flag indicating a content of the processing is recorded by appending the flag to the first image data.

7. The image processing apparatus according to claim 1 further comprising a display device,
wherein, in each of display areas of the display device predetermined according to the site, the first image data corresponding to the site based on the site information or second image data if there is the second image data obtained by processing the first image data by the processor is displayed as third image data.

8. The image processing apparatus according to claim 7, wherein at least one of the site information and the mirror use information of the first image data corresponding to the third image data is displayed in relation to the third image data in the display device, and
wherein the one or more processors and/or circuitry further comprises an input unit that is used to change the site information and the mirror use information displayed on the display device.

9. The image processing apparatus according to claim 7, wherein the one or more processors and/or circuitry further comprises a selector that selects the first image data which is image data before being processed by the processor, and
wherein, in a case where the first image data is selected by the selector, the first image data is displayed on the display device.

10. The image processing apparatus according to claim 7, wherein patient information and date and time of photography related to the first image data to which the third image data correspond is displayed on the display device.

11. An image capturing apparatus comprising:
an image sensor that takes intraoral photographs; and
one or more processors and/or circuitry which functions as:
a setting unit that sets site information indicating a site in an oral cavity, and mirror use information indicating whether or not a mirror was used at a time of photography; and
transmission unit that transmits to an external device an image file in which the site information and the mirror use information set at the time of photography are appended to image data of each of the intraoral photographs taken by the image sensor.

12. The image capturing apparatus according to claim 11 further comprising a memory that stores the site information and the mirror use information set by the setting unit.

13. An image capturing system comprising an image capturing apparatus and an image processing apparatus, wherein
the image capturing apparatus comprises:
an image sensor that takes intraoral photographs; and
one or more processors and/or circuitry which functions as:
a setting unit that sets site information indicating a site in an oral cavity, and mirror use information indicating whether or not a mirror was used at a time of photography; and
transmission unit that transmits to the image processing apparatus an image file in which the site information and the mirror use information set at the time of photography are appended to first image data of each of the intraoral photographs taken by the image sensor, and
the image processing apparatus comprises:
one or more processors and/or circuitry which functions as:
a receiving unit that receives the image file from the image capturing apparatus; and
a processor that determines whether or not it is necessary to perform at least one of horizontal reversal processing and vertical rotation processing on the first image data in the received image file based on the site information and the mirror use information, and performs the processing determined to be necessary.

14. An image processing apparatus comprising one or more processors and/or circuitry which functions as:
an acquisition unit that acquires image data of a plurality of intraoral photographs;
a determination unit that determines whether or not a site information indicating a site in an oral cavity, and mirror use information indicating whether or not a mirror was used at a time of photography are appended to the image data of each of the intraoral photographs acquired by the acquisition unit;
an inference unit that, in a case where neither of the site information and the mirror use information is appended, infers the site and whether or not a mirror was used based on a characteristic of each of the intraoral photographs; and
a memory that stores the site information and the mirror use information by appending them to the image data of each of the intraoral photographs based on the site and whether or not a mirror is used inferred by the inference unit.

15. The image processing apparatus according to claim 14, wherein the one or more processors and/or circuitry further comprises a processor that determines whether or not it is necessary to perform at least one of horizontal reversal processing and vertical rotation processing on the image data in an image file stored in the memory based on the site information and the mirror use information, and performs the processing determined to be necessary.

16. The image processing apparatus according to claim 14, wherein the inference unit performs
a first inference of inferring a site which appears in each of the intraoral photographs among a plurality of predetermined sites in an oral cavity based on a characteristic of each of the intraoral photographs, and
a second inference of inferring whether or not a mirror was used at a time of taking at least one of the intraoral photographs based on a condition of each tooth in the plurality of intraoral photographs inferred on the basis of the inferred site.

17. The image processing apparatus according to claim 16, wherein the sites are front, maxilla, mandible, right, and left in a five-sheet method, and
in the second inference, the inference unit
infers a condition of each tooth based on the intraoral photographs inferred to be of maxilla and mandible, infers a condition of each tooth based on the intraoral photographs inferred to be of right and left under assumption that no mirror was used at the time of photography, and infers a condition of each tooth based on the intraoral photographs inferred to be of right and left under assumption that a mirror was used at the time of photography, and
infers whether or not a mirror was used at the time of taking the intraoral photographs inferred to be of right and left based on consistency between the condition of each tooth in the maxilla and mandible and the condition of each tooth on the right and left under assumption that no mirror was used and consistency between the condition of each tooth in the maxilla and mandible and the condition of each tooth on the right and left under assumption that a mirror was used.

18. The image processing apparatus according to claim 14, wherein the inference unit infers the site appears in each of the intraoral photographs among a plurality of predetermined sites in an oral cavity based on a characteristic of each of the intraoral photographs.

19. The image processing apparatus according to claim 14, wherein the inference unit infers the site that appears in each of the intraoral photographs based on a detection result of a tissue other than teeth in each of the intraoral photographs.

20. The image processing apparatus according to claim 14, wherein the inference unit infers the site that appears in each of the intraoral photographs based on a detection result of types of teeth in each of the intraoral photographs.

21. The image processing apparatus according to claim 14, wherein the inference unit infers whether or not a mirror was used at the time of taking each of the intraoral photographs based on a detection result of a predetermined instrument in each of the intraoral photographs.

22. The image processing apparatus according to claim 14, wherein the inference unit infers whether or not a mirror was used at the time of taking each of the intraoral photographs based on a detection result of whether or not there is any tooth appeared outside a dentition in each of the intraoral photographs.

23. An image processing method comprising:
acquiring an image file including image data of an intraoral photograph, site information indicating a site in an oral cavity, and mirror use information indicating whether or not a mirror was used at a time of photography; and
determining whether or not it is necessary to perform at least one of horizontal reversal processing and vertical rotation processing on the image data in the image file based on the site information and the mirror use information, and performing the processing determined to be necessary.

24. An image capturing method comprising:
taking intraoral photographs; and
setting site information indicating a site in an oral cavity, and mirror use information indicating whether or not a mirror was used at a time of photography; and
transmitting to an external device an image file in which the site information and the mirror use information set at the time of photography are appended to image data of each of the intraoral photographs.

25. An image capturing method comprising:
in an image capturing apparatus,
taking intraoral photographs;
setting site information indicating a site in an oral cavity, and mirror use information indicating whether or not a mirror was used at a time of photography; and
generating an image file in which the site information and the mirror use information set at the time of photography are appended to image data of each of the intraoral photographs, and
in an image processing apparatus,
determining whether or not it is necessary to perform at least one of horizontal reversal processing and vertical rotation processing on the image data in the image file based on the site information and the mirror use information, and performing the processing determined to be necessary.

26. An image processing method comprising:
acquiring image data of a plurality of intraoral photographs;
determining whether or not a site information indicating a site in an oral cavity, and mirror use information indicating whether or not a mirror was used at a time of photography are appended to the image data of each of the intraoral photographs;
inferring, in a case where neither of the site information and the mirror use information is appended, the site and whether or not a mirror was used based on a characteristic of each of the intraoral photographs; and
storing the site information and the mirror use information by appending them to the image data of each of the intraoral photographs in a memory based on the inferred site and whether or not a mirror is used.

27. A non-transitory computer-readable storage medium, the storage medium storing a program that is executable by the computer, wherein the program includes program code for causing the computer to execute the image processing method comprising:
acquiring an image file including image data of an intraoral photograph, site information indicating a site in an oral cavity, and mirror use information indicating whether or not a mirror was used at a time of photography; and
determining whether or not it is necessary to perform at least one of horizontal reversal processing and vertical rotation processing on the image data in the image file based on the site information and the mirror use information, and performing the processing determined to be necessary.

28. A non-transitory computer-readable storage medium, the storage medium storing a program that is executable by the computer, wherein the program includes program code for causing the computer to execute the image capturing method comprising:
taking intraoral photographs; and
setting site information indicating a site in an oral cavity, and mirror use information indicating whether or not a mirror was used at a time of photography; and
transmitting to an external device an image file in which the site information and the mirror use information set at the time of photography are appended to image data of each of the intraoral photographs.

29. A non-transitory computer-readable storage medium, the storage medium storing a program that is executable by the computer, wherein the program includes program code for causing the computer to execute the image processing method comprising:
in the image capturing apparatus,
taking intraoral photographs;
setting site information indicating a site in an oral cavity, and mirror use information indicating whether or not a mirror was used at a time of photography; and
generating an image file in which the site information and the mirror use information set at the time of photography are appended to image data of each of the intraoral photographs, and
in the image processing apparatus,
determining whether or not it is necessary to perform at least one of horizontal reversal processing and vertical rotation processing on the image data in the image file based on the site information and the mirror use information, and performing the processing determined to be necessary.

30. A non-transitory computer-readable storage medium, the storage medium storing a program that is executable by the computer, wherein the program includes program code for causing the computer to execute the image processing method comprising:
acquiring image data of a plurality of intraoral photographs;
determining whether or not a site information indicating a site in an oral cavity, and mirror use information indicating whether or not a mirror was used at a time of photography are appended to the image data of each of the intraoral photographs;
inferring, in a case where neither of the site information and the mirror use information is appended, the site and whether or not a mirror was used based on a characteristic of each of the intraoral photographs; and
storing the site information and the mirror use information by appending them to the image data of each of the intraoral photographs in a memory based on the inferred site and whether or not a mirror is used.

31. A non-transitory computer-readable storage medium, the storage medium storing a program that is executable by the computer, wherein the program includes program code for causing the computer to function as an image processing apparatus comprising:
an acquisition unit that acquires an image file including first image data of an intraoral photograph, site information indicating a site in an oral cavity, and mirror use information indicating whether or not a mirror was used at a time of photography; and
a processor that determines whether or not it is necessary to perform at least one of horizontal reversal processing and vertical rotation processing on the first image data in the acquired image file based on the site information and the mirror use information, and performs the processing determined to be necessary.

32. A non-transitory computer-readable storage medium, the storage medium storing a program that is executable by the computer, wherein the program includes program code for causing the computer to function as an image processing apparatus comprising:
an acquisition unit that acquires image data of a plurality of intraoral photographs;
a determination unit that determines whether or not a site information indicating a site in an oral cavity, and mirror use information indicating whether or not a mirror was used at a time of photography are appended to the image data of each of the intraoral photographs acquired by the acquisition unit;
an inference unit that, in a case where neither of the site information and the mirror use information is appended, infers the site and whether or not a mirror was used based on a characteristic of each of the intraoral photographs; and
a memory that stores the site information and the mirror use information by appending them to the image data of each of the intraoral photographs based on the site and whether or not a mirror is used inferred by the inference unit.
